(19) United States
(12) Patent Application Publication
(10) Pub. No.: US 2024/0206901 A1
(43) Pub. Date: Jun. 27, 2024

(54) Portable Tongue Scraper Assembly

(71) Applicant: Samuel Areh, Fort Defiance, AZ (US)

(72) Inventor: Samuel Areh, Fort Defiance, AZ (US)

(21) Appl. No.: 18/085,984

(22) Filed: Dec. 21, 2022

(51) Int. Cl. A61B 17/24

(52) U.S. Cl. CPC A61B 17/244

(57) ABSTRACT

A portable tongue scraper assembly includes a housing that has a slot extending into an interior of the housing. A tongue scraper is pivotally disposed in the housing and the tongue scraper is positionable in a deployed position having the tongue scraper extending upwardly from the housing. The tongue scraper is positionable in a stored position having the tongue scraper being stored within the housing. A closure is pivotally disposed on the housing. The closure is positionable in a closed position when the tongue scraper is in the stored position having the closure closing the slot for enclosing the tongue scraper. The closure is positionable in an open position having the slot being exposed thereby facilitating the tongue scraper to be positioned in the deployed position.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to tongue scraper devices and more particularly pertains to a new tongue scraper device for facilitating oral hygiene to be performed while traveling. The device includes a housing with a slot extending longitudinally into said housing and a tongue scraper pivotally attached to the housing. The tongue scraper is positionable in a stored position or a deployed position. The device includes a closure pivotally attached to the housing for opening or closing the slot.

### (2) Description of Related Art Including Information Disclosed Under 37 CFR 1.97 and 1.98

The prior art relates to tongue scraper devices including a dental apparatus that includes a toothbrush that is attachable to a cylindrical housing. The prior art discloses a tongue scraper device that includes a tongue scraper being disposed on a toothbrush. The prior art discloses a tongue scraper that includes a head and a plurality of bristles attached to the head. The prior art discloses a tongue scraper that includes a member having a pair of textured lateral edges for scraping a tongue. The prior art discloses a tongue scraper that includes a plurality of curved members arranged to form a bulb on a handle.

### BRIEF SUMMARY OF THE INVENTION

An embodiment of the disclosure meets the needs presented above by generally comprising a housing that has a slot extending into an interior of the housing. A tongue scraper is pivotally disposed in the housing and the tongue scraper is positionable in a deployed position having the tongue scraper extending upwardly from the housing. The tongue scraper is positionable in a stored position having the tongue scraper being stored within the housing. A closure is pivotally disposed on the housing. The closure is positionable in a closed position when the tongue scraper is in the stored position having the closure closing the slot for enclosing the tongue scraper. The closure is positionable in an open position having the slot being exposed thereby facilitating the tongue scraper to be positioned in the deployed position.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of an embodiment of the disclosure showing a tongue scraper in a deployed position.

FIG. 2 is a perspective view of an embodiment of the disclosure showing a tongue scraper in a stored position.

FIG. 3 is a perspective view of a portable tongue scraper assembly according to an embodiment of the disclosure showing a cover in an open position and.

FIG. 4 is a front phantom view of an embodiment of the disclosure.

FIG. 5 is a left side phantom view of an embodiment of the disclosure.

FIG. 6 is a cross sectional view taken along line 6-6 of FIG. 2 of an embodiment of the disclosure.

FIG. 7 is a perspective view of an alternative embodiment of the disclosure.

FIG. 8 is an exploded perspective view of an alternative embodiment of the disclosure.

### DETAILED DESCRIPTION OF THE INVENTION

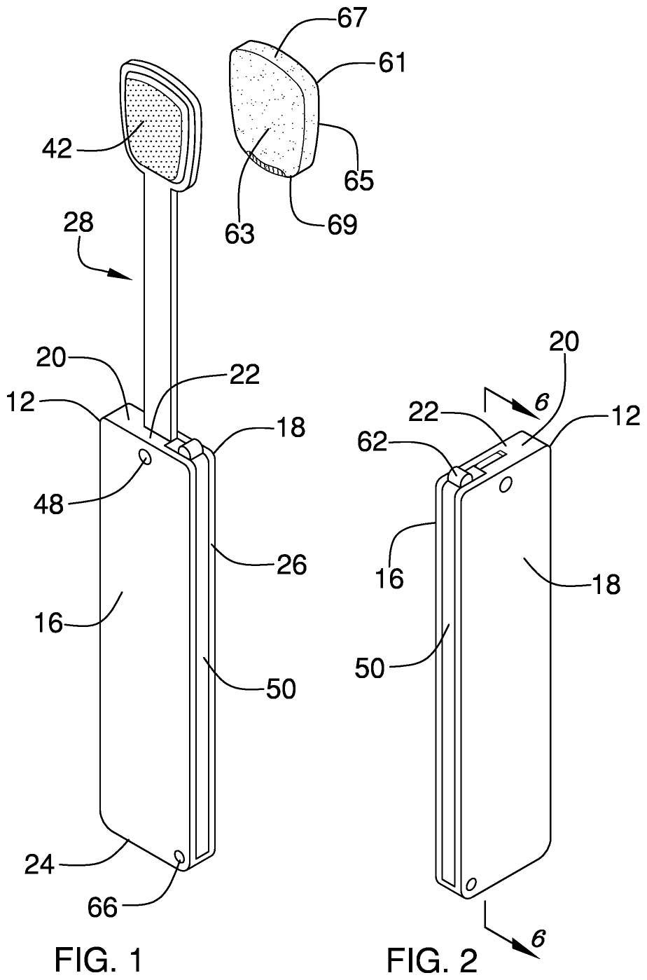

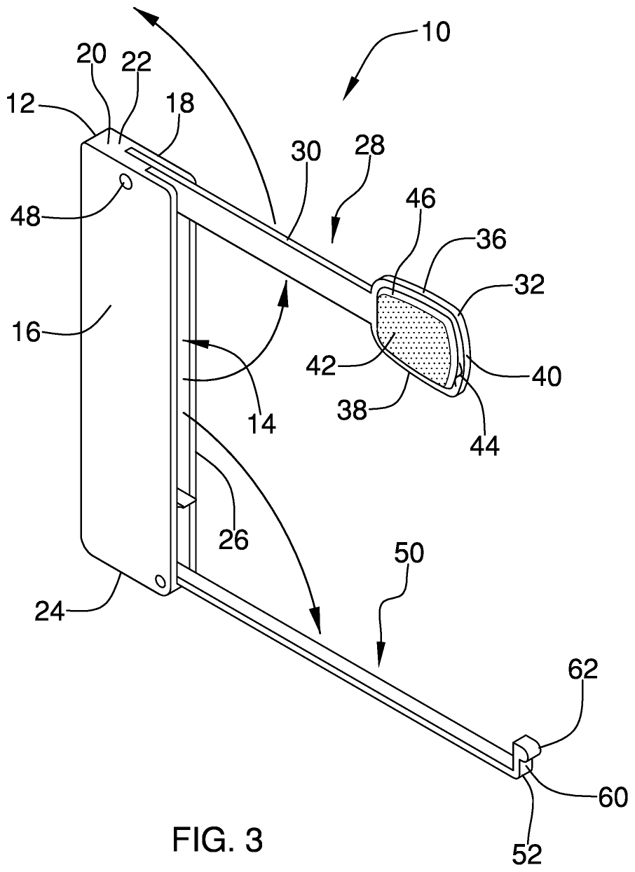

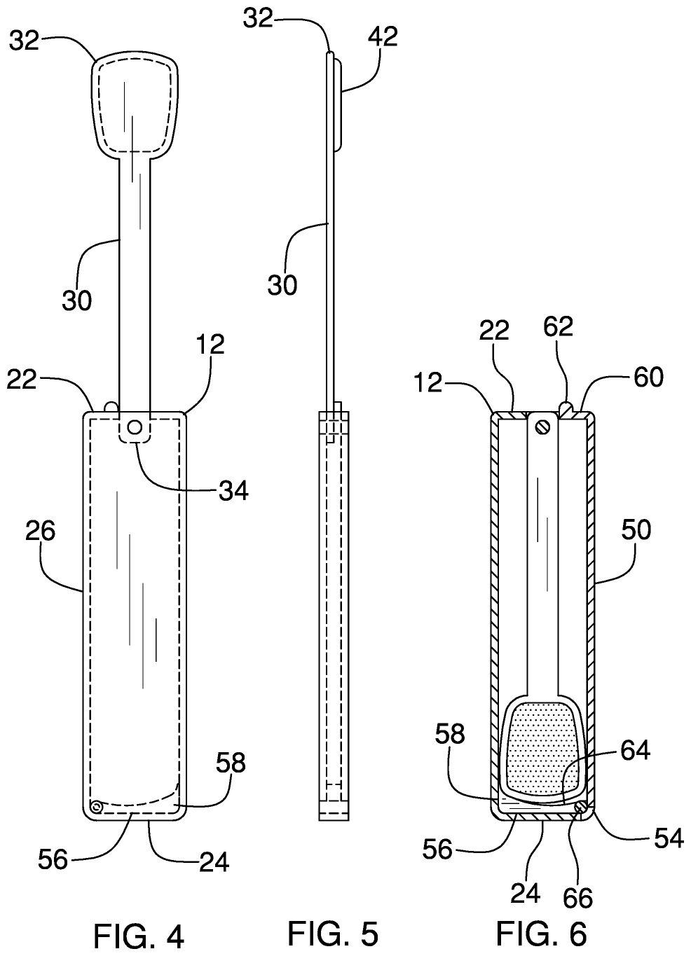

With reference now to the drawings, and in particular to FIGS. 1 through 8 thereof, a new tongue scraper device embodying the principles and concepts of an embodiment of the disclosure and generally designated by the reference numeral 10 will be described.

As best illustrated in FIGS. 1 through 8, the portable tongue scraper assembly 10 generally comprises a housing 12 that has a slot 14 extending into an interior of the housing 12. The housing 12 has a front wall 16, a back wall 18 and an outer wall 20 extending between the front wall 16 and the back wall 18 and the outer wall 20 has a top side 22, a bottom side 24 and a first lateral side 26. The housing 12 is elongated between the top side 22 and the bottom side 24. The slot 14 extends through a full height of the first lateral side 26 and the slot 14 extends through a substantial width of the top side 22.

A tongue scraper 28 is pivotally disposed in the housing 12 and the tongue scraper 28 is positionable in a deployed position having the tongue scraper 28 extending upwardly from the housing 12. Conversely, the tongue scraper 28 is positionable in a stored position having the tongue scraper 28 stored within the housing 12. The tongue scraper 28 is pivoted through the slot 14 in the housing 12 when the tongue scraper 28 is moved between the stored position and the deployed position. The tongue scraper 28 has a stem 30 and a head 32 and the stem 30 has a distal end 34 with respect to the head 32. The stem 30 extends outwardly through the slot 14 in the top side 22 of the outer wall 20 of the housing 12 when the tongue scraper 28 is in the deployed position such that the head 32 of the tongue scraper 28 is exposed. Conversely, the stem 30 and the head 32 are concealed within the housing 12 when the tongue scraper 28 is in the stored position.

The head 32 has a first sidelong side 36, a second sidelong side 38 and an upper side 40 and each of the first sidelong side 36 and the second sidelong side 38 angles outwardly between the stem 30 and the upper side 40 such that the head 32 has a trapezoidal shape. The head 32 has a scraping pad 42 that is applied to a forward surface 44 of the head 32. The scraping pad 42 has a perimeter edge 46 which follows the shape defined by the first sidelong side 36 and the second sidelong side 38 and the upper side 40 of the head 32. Additionally, the scraping pad 42 may be comprised of a textured and resiliently compressible material such as textured rubber or the like.

A first pin 48 extends through each of the front wall 16 and the back wall 18 of the housing 12 and the stem 30 of the tongue scraper 28 for pivotally attaching the tongue scraper 28 to the housing 12. Additionally, the first pin 48 engages the stem 30 at a point located adjacent to the distal end 34 of the stem 30. A closure 50 is provided and the closure 50 is pivotally disposed on the housing 12. The closure 50 is positionable in a closed position when the tongue scraper 28 is in the stored position having the closure 50 closing the slot 14 for enclosing the tongue scraper 28. Conversely, the closure 50 is positionable in an open position having the slot 14 being exposed thereby facilitating the tongue scraper 28 to be positioned in the deployed position.

The closure 50 has an upper end 52 and a lower end 54 and the closure 50 has a leg 56 extending away from the lower end 54 and a foot 58 extending upwardly from the leg 56 having the foot 58 being oriented parallel with the closure 50. The closure 50 has a tab 60 extending laterally away from the upper end 52 having the tab 60 being oriented parallel with the leg 56. The tab 60 has a lobe 62 extending upwardly from the tab 60 adjacent to a distal end 34 of the tab 60. Furthermore, the tab 60 extends into the slot 14 in the top side 22 of the outer wall 20 of the housing 12 when the closure 50 is in the closed position having the closure 50 extending along the slot 14 in the first lateral side 26 of the outer wall 20 of the housing 12 when the closure 50 is in the closed position. The tab 60 is displaced from the slot 14 in the top side 22 having the closure 50 being displaced from the slot 14 in the first lateral side 26 when the closure 50 is in the open position.

The lobe 62 extends upwardly from the top side 22 of the outer wall 20 when the closure 50 is in the closed position thereby facilitating the lobe 62 to be gripped for urging the closure 50 into the open position. An upper edge 64 of the leg 56 is concavely arcuate between the closure 50 and the foot 58 thereby conforming to the upper side 40 of the head 32 when the tongue scraper 28 is in the stored position and the closure 50 is in the closed position. Moreover, the foot 58 urges the head 32 outwardly through the slot 14 when the closure 50 is urged into the open position thereby facilitating the tongue scraper 28 to be gripped for fully positioning the tongue scraper 28 in the deployed position. A second pin 66 extends through the front wall 16 and the back wall 18 and the closure 50 at an intersection between the leg 56 and the closure 50 for pivotally attaching the closure 50 to the housing 12.

As is most clearly shown in FIG. 1, a cover 61 is provided that has a forward wall 63, a rearward wall 65 and an exterior wall 67 extending between the forward wall 63 and the rearward wall 65. A lower side 69 of the exterior wall 67 is open for insertably receiving the head 32 of the tongue scraper 28. The exterior wall 67 is flared such that the cover 61 conforms to the shape of the head 32. Additionally, the cover 61 is comprised of a fluid impermeable material, including but not being limited to rubber or silicone, thereby protecting the head 32 from contamination.

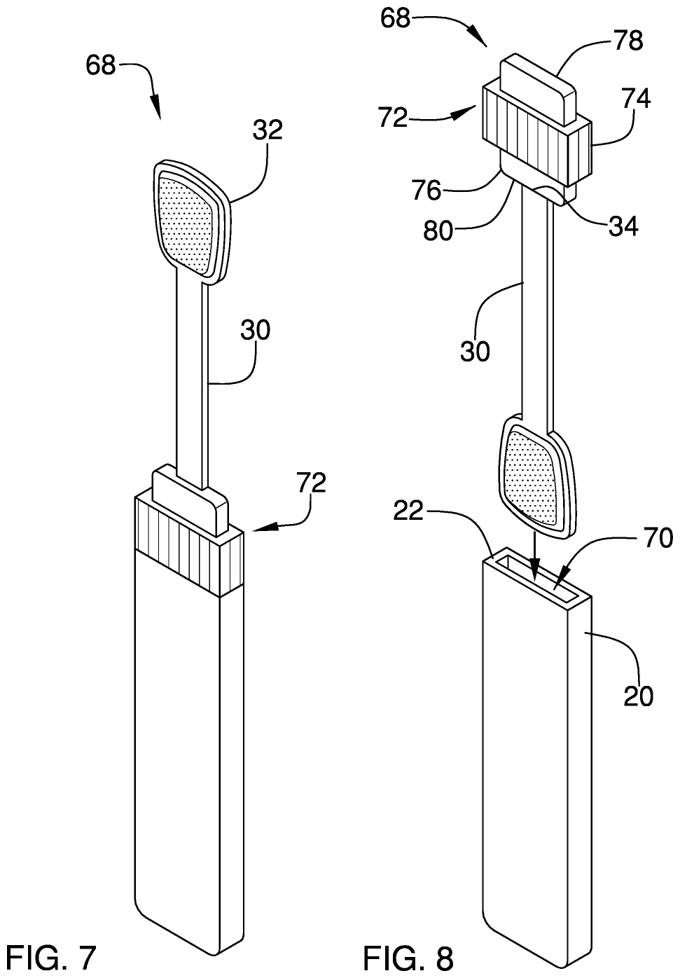

In an alternative embodiment 68 as is most clearly shown in FIGS. 7 and 8, the top side 22 of the outer wall 20 of the housing 12 has an opening 70 extending into an interior of the housing 12. The tongue scraper 28 includes a mating unit 72 that is attached to the distal end 34 of the stem 30. The mating unit 72 includes a block 74 extending between an upper lobe 76 and a lower lobe 78 and the distal end 34 of the stem 30 is attached to an upper edge 80 of the upper lobe 76. The lower lobe 78 is insertable into the opening 70 in the top side 22 of the outer wall 20 of the housing 12 having the block 74 resting against the top side 22 when the tongue scraper 28 is positioned in the deployed position. Conversely, the head 32 and the stem 30 are extended through the opening 70 having the upper lobe 76 being inserted into the opening 70 in the top side 22 of the outer wall 20 of the housing 12 when the tongue scraper 28 is positioned in the stored position.

In use, the lobe 62 is gripped to urge the closure 50 into the open position thereby urging the head 32 of the tongue scraper 28 outwardly from the housing 12. The tongue scraper 28 is pivoted into the deployed position and the closure 50 is positioned in the closed position. In this way the tongue scraper 28 can be employed for oral hygiene purposes. The lobe 62 is gripped to urge the closure 50 into the open position, the tongue scraper 28 is pivoted into the stored position and the closure 50 is urged into the closed position. In this way the tongue scraper 28 is stored within the housing 12 when the tongue scraper 28 is not being employed.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A portable tongue scraper assembly for facilitating a user to scrape their tongue while traveling, said assembly comprising:
a housing having a slot extending into an interior of said housing;
a tongue scraper being pivotally disposed in said housing, said tongue scraper being positionable in a deployed position having said tongue scraper extending upwardly from said housing, said tongue scraper being positionable in a stored position having said tongue scraper being stored within said housing having said tongue scraper being pivoted through said slot in said housing; and
a closure being pivotally disposed on said housing, said closure being positionable in a closed position when said tongue scraper is in said stored position having said closure closing said slot for enclosing said tongue scraper, said closure being positionable in an open position having said slot being exposed thereby facilitating said tongue scraper to be positioned in said deployed position.

2. The assembly according to claim 1, wherein:
said housing has a front wall, a back wall and an outer wall extending between said front wall and said back wall, said outer wall having a top side, a bottom side and a first lateral side, said housing being elongated between said top side and said bottom side, said slot extending through said first lateral side, said slot extending through a substantial width of said top side; and
said tongue scraper has a stem and a head, said stem having a distal end with respect to said head, said stem extending outwardly through said slot in said top side of said outer wall of said housing when said tongue scraper is in said deployed position such that said head of said tongue scraper is exposed, said stem and said head being concealed within said housing when said tongue scraper is in said stored position.

3. The assembly according to claim 2, wherein said head has a first sidelong side, a second sidelong side and an upper side, each of said first sidelong side and said second sidelong side angling outwardly between said stem and said upper side such that said head has a trapezoidal shape, said head having a scraping pad being applied to a forward surface of said head, said scraping pad having a perimeter edge which follows the shape defined by said first sidelong side and said second sidelong side and said upper side of said head.

4. The assembly according to claim 2, further comprising a first pin extending through each of said front wall and said back wall of said housing and said stem of said tongue scraper for pivotally attaching said tongue scraper to said housing, said first pin engaging said stem at a point located adjacent to said distal end of said stem.

5. The assembly according to claim 2, wherein:
said closure has an upper end and a lower end;
said closure has a leg extending away from said lower end and a foot extending upwardly from said leg having said foot being oriented parallel with said closure;
said closure has a tab extending laterally away from said upper end having said tab being oriented parallel with said leg;
said tab has a lobe extending upwardly from said tab adjacent to a distal end of said tab;
said tab extends into said slot in said top side of said outer wall of said housing when said closure is in said closed position having said closure extending along said slot in said first lateral side of said outer wall of said housing when said closure is in said closed position;
said tab is displaced from said slot in said top side having said closure being displaced from said slot in said first lateral side when said closure is in said open position; and
said lobe extends upwardly from said top side of said outer wall when said closure is in said closed position thereby facilitating said lobe to be gripped for urging said closure into said open position.

6. The assembly according to claim 5, wherein an upper edge of said leg is concavely arcuate between said closure and said foot thereby conforming to said upper side of said head when said tongue scraper is in said stored position and said closure is in said closed position;

7. The assembly according to claim 5, wherein said foot urges said head outwardly through said slot when said closure is urged into said open position thereby facilitating said tongue scraper to be gripped for fully positioning said tongue scraper in said deployed position.

8. The assembly according to claim x, further comprising a second pin extending through said front wall and said back wall and said closure at an intersection between said leg and said closure for pivotally attaching said closure to said housing.

9. A portable tongue scraper assembly for facilitating a user to scrape their tongue while traveling, said assembly comprising:
a housing having a slot extending into an interior of said housing, said housing having a front wall, a back wall and an outer wall extending between said front wall and said back wall, said outer wall having a top side, a bottom side and a first lateral side, said housing being elongated between said top side and said bottom side, said slot extending through said first lateral side, said slot extending through a substantial width of said top side;
a tongue scraper being pivotally disposed in said housing, said tongue scraper being positionable in a deployed position having said tongue scraper extending upwardly from said housing, said tongue scraper being positionable in a stored position having said tongue scraper being stored within said housing having said tongue scraper being pivoted through said slot in said housing, said tongue scraper having a stem and a head, said stem having a distal end with respect to said head, said stem extending outwardly through said slot in said top side of said outer wall of said housing when said tongue scraper is in said deployed position such that said head of said tongue scraper is exposed, said stem and said head being concealed within said housing when said tongue scraper is in said stored position, said head having a first sidelong side, a second sidelong side and an upper side, each of said first sidelong side and said second sidelong side angling outwardly between said stem and said upper side such that said head has a trapezoidal shape, said head having a scraping pad being applied to a forward surface of said head, said scraping pad having a perimeter edge which follows the shape defined by said first sidelong side and said second sidelong side and said upper side of said head;
a first pin extending through each of said front wall and said back wall of said housing and said stem of said tongue scraper for pivotally attaching said tongue scraper to said housing, said first pin engaging said stem at a point located adjacent to said distal end of said stem;
a closure being pivotally disposed on said housing, said closure being positionable in a closed position when said tongue scraper is in said stored position having said closure closing said slot for enclosing said tongue scraper, said closure being positionable in an open position having said slot being exposed thereby facilitating said tongue scraper to be positioned in said deployed position, said closure having an upper end and a lower end, said closure having a leg extending away from said lower end and a foot extending upwardly from said leg having said foot being oriented parallel with said closure, said closure having a tab extending laterally away from said upper end having said tab being oriented parallel with said leg, said tab having a lobe extending upwardly from said tab adjacent to a distal end of said tab, said tab extending into said slot in said top side of said outer wall of said housing when said closure is in said closed position having said closure extending along said slot in said first lateral side of said outer wall of said housing when said closure is in said closed position, said tab being displaced from said slot in said top side having said closure being displaced from said slot in said first lateral side when said closure is in said open position, said lobe extending upwardly from said top side of said outer wall when said closure is in said closed position thereby facilitating said lobe to be gripped for urging said closure into said open position, an upper edge of said leg being concavely arcuate between said closure and said foot thereby conforming to upper side of said head when said tongue scraper is in said stored position and said closure is in said closed position, said foot urging said head outwardly through said slot when said closure is urged into said open position thereby facilitating said tongue scraper to be gripped for fully positioning said tongue scraper in said deployed position; and
a second pin extending through said front wall and said back wall and said closure at an intersection between said leg and said closure for pivotally attaching said closure to said housing.

10. The assembly according to claim 9, wherein:
top side of said outer wall of said housing has an opening extending into an interior of said housing; and
said tongue scraper includes a mating unit being attached to said distal end of said stem, said mating unit includes a block extending between an upper lobe and a lower lobe, said distal end of said stem being attached to an upper edge of said upper lobe, said lower lobe being insertable into said opening in said top side of said outer wall of said housing having said block resting against said top side when said tongue scraper is positioned in said deployed position, said head and said stem being extended through said opening having said upper lobe being inserted into said opening said top side of said outer wall of said housing when said tongue scraper is positioned in said stored position.
